(19) United States
(12) Patent Application Publication
(10) Pub. No.: US 2024/0115391 A1
(43) Pub. Date: Apr. 11, 2024

(54) TOTAL ELBOW REPLACEMENT PROSTHESIS

(71) Applicant: Fitzbionics Limited, Godalming, Surrey (GB)

(72) Inventors: Noel Fitzpatrick, Godalming, Surrey (GB); Jayantilal Meswania, Godalming, Surrey (GB)

(73) Assignee: Fitzbionics Limited, Godalming, Surrey (GB)

(21) Appl. No.: 18/264,599

(22) PCT Filed: Feb. 11, 2022
(86) PCT No.: PCT/GB2022/050382

§ 371 Date: Aug. 8, 2023

(30) Foreign Application Priority Data
Feb. 11, 2021 (GB) 2101910.4

(51) Int. Cl. A61F 2/38

(52) U.S. Cl. CPC A61F 2/3804; A61F 2002/30131; A61F 2002/3818; A61F 2002/3822

(57) ABSTRACT

A total elbow replacement prosthesis (100) comprising: a radioulnar component (20) having an ulnar bearing surface (22) and an anconeal process bearing surface (24), a humeral component (40) having a trochlear bearing surface (42), the trochlear bearing surface being configured for bearing against the ulnar bearing surface, the humeral component further comprising an olecranon aperture boundary bearing surface (50), the anconeal process bearing surface being configured for bearing against the olecranon aperture boundary bearing surface.

### CROSS REFERENCE TO RELATED APPLICATIONS

The present application is the U.S. national stage application of international application PCT/GB2022/050382 filed Feb. 11, 2022, which international application was published on Aug. 18, 2022 as International Publication WO 2022/172029 A1. The international application claims priority to British Patent Application No. 2101910.4 filed Feb. 11, 2021.

### FIELD OF THE INVENTION

The invention relates to a total elbow replacement prosthesis for resurfacing bone at the elbow joint of a human or animal subject. The invention also relates to kits for a total elbow replacement prosthesis assembly and methods for manufacturing a total elbow replacement prosthesis.

### BACKGROUND TO THE INVENTION

Elbow dysplasia is the most common cause of forelimb lameness in young, large and giant breed dogs. Collectively, elbow dysplasia and elbow osteoarthritis are the commonest causes of forelimb lameness in dogs of any age. Small dogs can also be affected by elbow dysplasia. The clinical impact of elbow osteoarthritis is unpredictable and, regardless of treatment, arthritis will progress to some extent for all affected joints. In some dogs, lameness can be mild and intermittent, whilst in others, lameness can cause severe and permanent disability. Where persistent cartilage erosion occurs, it is generally in the inner (medial) part of the elbow.

In a natural canine elbow, being a quadruped animal, the elbow provides a simple hinge configuration for flexion/extension of the limb together with a torsional load transfer capability generated by the ground contact forces during walking, running, twisting and turning motions. To enable these forces the joint has evolved with a series of complex articular surfaces.

Elbow implants are available for resurfacing the joint. In some cases a unicompartmental implant may be employed, which replaces one compartment of the joint (i.e. the medial compartment). In other cases a total elbow replacement implant is needed. Total elbow replacement (TER) implants are available for canine subjects, however the existing total elbow replacements have various deficiencies. An improved Total elbow replacement (TER) implant is therefore sought.

### SUMMARY OF THE INVENTION

According to a first aspect of the invention there is provided a total elbow replacement prosthesis comprising: a radioulnar component having an ulnar bearing surface and an anconeal process bearing surface, a humeral component having a trochlear bearing surface, the trochlear bearing surface being configured for bearing against the ulnar bearing surface, the humeral component further comprising an olecranon aperture boundary bearing surface, the anconeal process bearing surface being configured for bearing against the olecranon aperture boundary bearing surface.

The olecranon aperture boundary bearing surface is suitably shaped to resurface at least a portion of the boundary of the olecranon aperture of a subject. The term olecranon aperture boundary bearing surface is synonymous with an olecranon fossa boundary bearing surface, should the subject merely have a depression rather than a full window through the bone. The total elbow replacement prosthesis is preferably configured for implantation in canine subjects.

Preferably the olecranon aperture boundary bearing surface is shaped to resurface at least a portion of the lateral boundary of the olecranon aperture of a subject. The olecranon aperture boundary bearing surface is preferably shaped to resurface at least a portion of the lateral side and base of the boundary of the olecranon aperture of a subject.

In preferred embodiments the olecranon aperture boundary bearing surface and the anconeal process bearing surface are both sized and shaped for bearing against one another, once implanted, when the elbow joint is in full extension. The shape and size of each of the humeral component and radioulnar component is predetermined such that the trochlear bearing surface bears against the ulnar bearing surface in use and the anconeal process bearing surface bears against the olecranon aperture boundary surface in use when the joint is in full extension. The thickness of each of the humeral component and radioulnar component from its back surface to its front surface may be predetermined such that the trochlear bearing surface bears against the ulnar bearing surface in use and the anconeal process bearing surface bears against the olecranon aperture boundary surface in use.

In preferred embodiments the olecranon aperture boundary bearing surface of the humeral component is shaped and sized to be seated on a subject's prepared bone surface at the olecranon aperture boundary (or the olecranon fossa if the subject does not have an aperture through the distal end of the humerus). Similarly, the anconeal process bearing surface is sized and shaped to be seated on a subject's prepared bone surface at the anconeal process. In this way, the bone to be removed from the subject is minimised. In preferred embodiments the total elbow replacement prosthesis preserves the subject's native olecranon aperture (or the olecranon fossa if the subject does not have an aperture through the distal end of the humerus) and native anconeal process. Advantageously the native olecranon aperture boundary and anconeal process do not need to be fully osteotomized in order to implant the prosthesis. The olecranon aperture boundary bearing surface and the anconeal process bearing surface of the total elbow replacement prosthesis merely resurface the prepared native bone of the subject at the olecranon aperture and anconeal process.

The humeral component and the radioulnar component are preferably handed and are provided in right and left handed versions, depending whether for implantation at a left or right elbow. The right and left handed versions of the humeral component are mirror images of one another, as are the right and left handed versions of the radioulnar component.

Preferably the olecranon aperture boundary bearing surface comprises a lateral side wall upstanding from the humeral component.

Preferably the olecranon aperture boundary bearing surface comprises a base and a lateral side wall upstanding from the base, the olecranon aperture boundary bearing surface being configured such that the base blends into the lateral side wall. The base of the olecranon aperture boundary bearing surface preferably has a substantially concave cylindrical form and blends into the lateral side wall. Suitably the base of the olecranon aperture boundary bearing surface blends curvedly into the lateral side wall.

Preferably the trochlear bearing surface is comprised on a main portion of the humeral component, the main portion comprising an elongate member with an outer surface of substantially C-shaped cross-section, the radius of the outer surface of the main portion varying along the elongate axis of the trochlear bearing surface. The olecranon aperture boundary bearing surface is preferably formed on a tail portion of the humeral component which extends from the trochlear bearing surface of the humeral component.

Preferably the trochlear bearing surface is comprised on a main portion of the humeral component, the main portion being an elongate member with an outer surface, the outer surface of the main portion being substantially bobbin shaped.

Preferably the trochlear bearing surface comprises a medial condylar bearing surface and a lateral condylar bearing surface, each for bearing against a corresponding depression in the ulnar bearing surface. The trochlear bearing surface comprises the medial condylar bearing surface and a lateral condylar bearing surface, separated by a groove therebetween. The groove corresponds to the isthmus of the natural humeral articular bone. The medial condylar bearing surface and a lateral condylar articular surface each comprise a bulbous articular bearing surface.

The humeral component preferably comprises means for attaching it to a humerus bone. Preferably the humeral component is stemless. In other words, the humeral component has no intramedullary stem for implanting in the intramedullary canal of a humerus bone.

Preferably the humeral component has an outer surface which is generally convex in the sagittal plane.

Preferably the olecranon aperture boundary bearing surface of the humeral component has an outer surface which is generally concave in the coronal plane.

Preferably the radioulnar component has an outer surface which is generally concave in the sagittal plane.

Preferably the ulnar bearing surface blends into the anconeal process bearing surface. Suitably the ulnar bearing surface blends curvedly into the anconeal process bearing surface.

Preferably the radioulnar component comprises means for attaching it to a subject's ulna bone and to the subject's radius bone.

Preferably the prosthesis further comprising an augmentation plate for attachment to a subject's radius and ulna.

According to a further aspect of the invention there is also provided a method of implanting a total elbow replacement prosthesis according to any aspect of the invention as described herein.

According to a further aspect of the invention there is also provided a method of manufacturing a total elbow replacement prosthesis according to any aspect of the invention as described herein, the method comprising making a radioulnar component having an ulnar bearing surface and an anconeal process bearing surface, making a humeral component having a trochlear bearing surface, the trochlear bearing surface being configured for bearing against the ulnar bearing surface, the humeral component further comprising an olecranon aperture boundary bearing surface, the anconeal process bearing surface being configured for bearing against the olecranon aperture boundary bearing surface.

In preferred embodiments the method further comprises shaping and sizing the humeral component and the radioulnar component such that the olecranon aperture boundary bearing surface and the anconeal process bearing surface bear against one another, once implanted in a subject's elbow joint, when the elbow joint is in full extension.

As used herein, the term “comprising” means the presence of the stated features, integers, steps, or components as referred to in the claims, but that it does not preclude the presence or addition of one or more other features, integers, steps, components or groups thereof.

The term subject as used herein can be a human or animal subject.

The terms lateral, anterior, posterior, caudal, sagittal, coronal etc as used herein have the usual meanings in relation to anatomy. Anatomical directional terms used herein in relation to the assembly or components of the assembly refer to anatomical planes/axes of the assembly when the assembly or components of the assembly is/are installed in a subject. It will be understood that components of the invention can be positioned in a number of different orientations when outside of the subject, the directional terminology being used for purposes of illustration.

### BRIEF DESCRIPTION OF THE DRAWINGS

A preferred embodiment of the present invention will now be more particularly described by way of example only with reference to the accompanying drawings, wherein:

FIG. 1 shows a side view of a total elbow replacement (TER) prosthesis assembly according to the invention installed in a subject;

FIG. 2 shows a posterior/caudal view of humeral component of the (TER) prosthesis assembly;

FIG. 3 shows a perspective view of the humeral component;

FIG. 4 shows a further perspective view of the humeral component;

FIG. 5 shows an underside view of perspective view of the humeral component;

FIGS. 6 to 8 show three different views of the bearing insert for the radioulnar component of the (TER) prosthesis assembly;

FIGS. 9 to 11 show three different views of the supporting body, often referred to as a tray, for the radioulnar component of the (TER) prosthesis assembly;

FIG. 12 shows the bearing insert of the radioulnar component;

FIG. 13 shows the supporting body of the radioulnar component;

FIG. 14 shows the radioulnar component;

FIG. 15 shows the humeral component;

FIG. 16 shows the radioulnar component;

FIG. 17 shows the humeral component and radioulnar component assembled together;

FIG. 18 shows a caudal view of the distal end of the subject's humerus;

FIGS. 19 to 21 show three different view of the subject's humerus with the humeral component installed;

FIGS. 22 and 23 show two different views of the subject's humerus with the humeral component and radioulnar component installed;

FIG. 24 shows the humeral component;

FIG. 25 shows the radioulnar component;

FIG. 26 shows the humeral component and radioulnar component assembled together;

FIG. 27 shows the radioulnar component installed on a subject's bone;

FIGS. 28 and 29 show two different view of the subject's humerus with the humeral component installed;

FIGS. 30 and 31 show two different views of the subject's elbow joint with the (TER) prosthesis assembly installed;

FIG. 32 shows a subject's ulna

FIGS. 33 to 37 show an alternative embodiment of a TER prosthesis assembly according to the invention wherein FIG. 33 shows the humeral component;

FIG. 34 shows the humeral component and radioulnar component assembled together;

FIG. 35 shows an alternative view of the humeral component;

FIG. 36 shows an alternative view of the humeral component and radioulnar component assembled together; and

FIG. 37 shows the radioulnar component.

### DESCRIPTION OF THE PREFERRED EMBODIMENTS

The present embodiments represent currently the best ways known to the applicant of putting the invention into practice. But they are not the only ways in which this can be achieved. They are illustrated, and they will now be described, by way of example only.

Referring to the Figures, a total elbow replacement prosthesis assembly 100 is shown for replacing an elbow joint. The assembly shown in the Figures is suitable for replacing a right elbow joint in a quadruped, however it will be understood that the features are also suitable for an assembly for implantation in an elbow joint in a biped and that the features can be incorporated into a left elbow joint replacement assembly (being a mirror image of the right elbow replacement prosthesis assembly). The prosthesis assembly 100 is especially suited for implantation in dogs, however similar designs may be envisaged for humans or other animals.

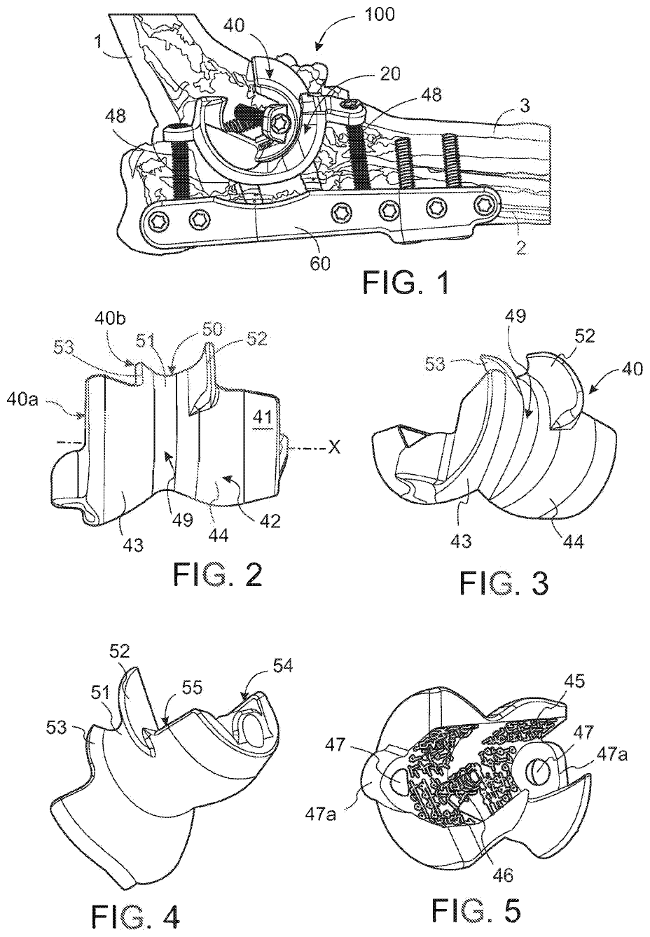

Referring to FIGS. 1, 18 and 32, the bones of the arm comprise the humerus 1, the ulna 2 and the radius 3. At the elbow the humerus 1 widens to its lateral and medial epicondyles 7 and 9 and is provided at its end with articulating surfaces on the trochlea 11 and the capitellum 13. These articulating surfaces engage with corresponding surfaces on the coronoid process of the ulna (at the trochlear notch 5) and the head of the radius.

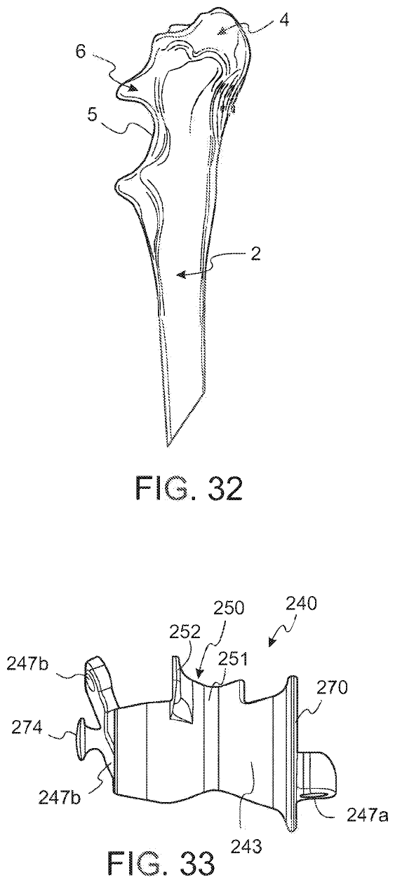

Referring to FIG. 18, the olecranon aperture 10 is a window of the humerus that communicates the olecranon fossa with the coronoid fossa. It is sometimes also known as the supratrochlear foramen. Referring to FIG. 32, in full extension, the anconeal process 6, which is on the olecranon 4 of the ulna 2, is received by the olecranon aperture 10 on the posterior aspect of the humerus. Typically in humans, there is no olecranon aperture, but merely depressions on the posterior and anterior sides of the bone, namely the olecranon fossa and the coronoid fossa. In humans the anconeal process of the ulna is received by the olecranon fossa on the posterior aspect of the humerus. However in dogs and cats there is typically an olecranon aperture 10 communicating between the olecranon fossa with the coronoid fossa.

Referring to FIG. 1, a total elbow replacement prosthesis 100 assembly is shown. The prosthesis assembly comprises a radioulnar component 20 for attachment to the ulna and radius and for resurfacing the ulna and a humeral component 40 for resurfacing the humerus at the elbow joint. Once implanted, the radioulnar component 20 and humeral component 40 articulate with one another to restore function at the joint.

Referring to FIG. 1, the radioulnar component 20 is configured for attaching to the heads of the ulna and radius of a subject, thus fusing the radius and ulna bones together. Referring to FIG. 7, the radioulnar component 20 has a contoured outer surface 21, configured to face away from the ulna when implanted, defining an ulnar bearing surface 22 and an anconeal process bearing surface 24. The ulnar bearing surface 22 is configured for resurfacing the trochlear notch of a subject's ulna. The anconeal process bearing surface 24 is configured for resurfacing the anconeal process of the ulna. The radioulnar component 20 is not configured to recreate the anatomical shape of the articular surface on the subject's radial head. Therefore the embodiment in the figures does not recreate the radiohumeral joint in the present prosthesis assembly as such. In other embodiments however, the anatomical shape of the radiohumeral joint may be preserved by an alternative prosthesis design.

Referring to FIG. 2, the humeral component 40 has a contoured outer surface 41 defining an articular surface comprising a trochlear bearing surface 42 and an olecranon aperture boundary bearing surface 50. The trochlear bearing surface 42 comprises first and second bulbous bearing surfaces which we term herein the medial condylar bearing surface 43 and lateral condylar bearing surface 44. The trochlear bearing surface 42 is formed on a main portion 40a of the humeral component, the olecranon aperture boundary bearing surface 50 being on a tail portion 40b which projects from the main portion 40a. The trochlear bearing surface 42 is a major or primary bearing surface of the humeral component, the olecranon aperture boundary bearing surface 50 having a smaller surface area than the trochlear bearing surface 42, therefore the olecranon aperture boundary bearing surface 50 forming a minor or secondary bearing surface.

Referring to FIG. 2, the main portion 40a of the humeral component is elongate, having an elongate axis X, the outer surface 41 of the main portion having a substantially C-shaped or arc shaped cross-section, the radius of the outer surface varying along the elongate axis of the main portion. The varying radial cross-sectional shape along its axis X forms an outer surface 41 having a shape akin a bobbin (i.e. a trochleiform shape). In preferred embodiments such as the assembly in the Figures, the bobbin shape is asymmetrical. The bobbin shaped outer surface comprises first and second bulbous portions at its medial and lateral sides, the first and second bulbous portions being divided by a groove 49 therebetween corresponding to the isthmus of the articular surface of a humeral bone. The outer surface of the medial bulbous portion defines the medial condylar bearing surface 43 and the outer surface of the lateral bulbous portion defines the lateral condylar bearing surface 44. The radial cross-section of the medial bulbous portion is larger at its maximum radial size than the radial cross-section of the lateral bulbous portion at its lateral radial size. The medial condylar bearing surface 43 and lateral condylar bearing surface 44 are configured for bearing against corresponding depressions in the ulnar bearing surface 22 respectively.

The tail portion 40b projecting from the main portion 40a of the humeral component has a generally concave articular surface in the coronal plane, defining the olecranon aperture boundary bearing surface 50. Referring to FIGS. 18 and 19, the olecranon aperture boundary bearing surface 50 is configured for resurfacing a portion 10a of the boundary of the olecranon aperture 10 of a subject (or the olecranon fossa if the subject does not have an aperture through the distal end of the humerus). In the preferred embodiment in the figures, the olecranon aperture boundary bearing surface 50 resurfaces a portion of the base 10aa and the lateral side 10ab of the boundary of the olecranon aperture of a subject. In this way the olecranon aperture boundary bearing surface 50 supports the anconeal process bearing surface 24 of the radioulnar component 20 as it enters and exits the olecranon aperture 10 (or the olecranon fossa) of the humerus during extension and flexion of the elbow joint.

The olecranon aperture boundary bearing surface 50 comprises a base 51 and a lateral side wall 52 upstanding from the base. The base 51 is generally concave and continues the profile of the groove 49 of the main portion 40a of the humeral component 40. The lateral side wall 52 projects from the lateral side of the base 51, the olecranon aperture boundary bearing surface being configured such that the base 51 blends curvedly into the lateral side wall 52. The olecranon aperture boundary bearing surface 50 further comprises a medial side wall 53 projecting from the medial side of the base 51, however the medial side wall 53 is shorter than the lateral side wall 52. The overall profile of the outer surface of the olecranon aperture boundary bearing surface 50 is generally concave in the coronal plane, having a lateral wing formed by the lateral side wall 52. The olecranon aperture boundary bearing surface 50 is shaped to provide a curved track for supporting the anconeal process bearing surface 24 of the radioulnar component 20 during extension and flexion of the joint. The curved axis of the track of the olecranon aperture boundary bearing surface 50 continues the arc formed by groove 49 of the main portion 40a of the humeral component 40. The curved axis of the track of the olecranon aperture boundary bearing surface 50 is formed in a plane that is orthogonal to the elongate axis X of the main portion 40a of the humeral component 40. The humeral component has an anterior end 54 and a posterior end 55, the humeral component being configured to be implanted with its anterior end 54 directed towards the anterior side of the humerus and its posterior end 55 directed towards the posterior side of the humerus. In the preferred embodiment in the figures, the groove 49 runs from the anterior end 54 to the posterior end 55 of the humeral component. In alternative embodiments the groove 49 may run at least part of the way from the anterior end 54 to the posterior end 55 of the humeral component, passing through both the trochlear bearing surface and the olecranon aperture boundary bearing surface.

The humeral component 40 is preferably made of metal. In preferred embodiments, the humeral component 40 is made of cobalt chrome. Referring to FIG. 5, the humeral component 40 has an inner surface 45 for seating against a prepared implantation site on the distal end of a subject's humerus. The inner surface 45 is generally concave in shape for seating against the convex bone surface of the distal humerus. The inner surface 45 has a textured surface to promote attachment to the prepared bone surface. For example, the textured inner surface 45 may be porous or stippled. The textured surfaces of the joint assembly are preferably coated with hydroxyapatite (HA) to further promote bone attachment. Treatments other than surface texture and/or HA coating can of course be provided on the humeral component to promote attachment to the bone.

Referring to FIG. 5, the humeral component 40 is stemless, in that it does not have an intramedullary stem for receipt within the intramedullary canal of a subject's humerus. As the humeral component 40 is stemless, it is also forkless unlike some stemmed earlier TER prosthesis designs, in that it does not have any fork at the base of any intramedullary stem for providing a widened access to the olecranon aperture. The humeral component 40 has a small post 46 projecting from its inner surface 45. The post 46 is a fixation post arranged to secure the humeral component 40 in place on the humerus. Optionally, other fixation designs may be used, including multiple posts, ribs or blades. The humeral component 40 comprises first and second through holes 47 at its medial and lateral sides respectively, each for receiving a screw 48 (visible in FIGS. 1, 30 and 31) for securing the humeral component to the bone. Optionally, other fixation designs may be used other than screw fixation. The medial screw hole 47 is located in a wing 47a which projects from the medial side of the humeral component, and similarly the lateral screw hole 47 is located in a wing 47a which projects from the lateral side of the humeral component, however screw holes may be located at other positions.

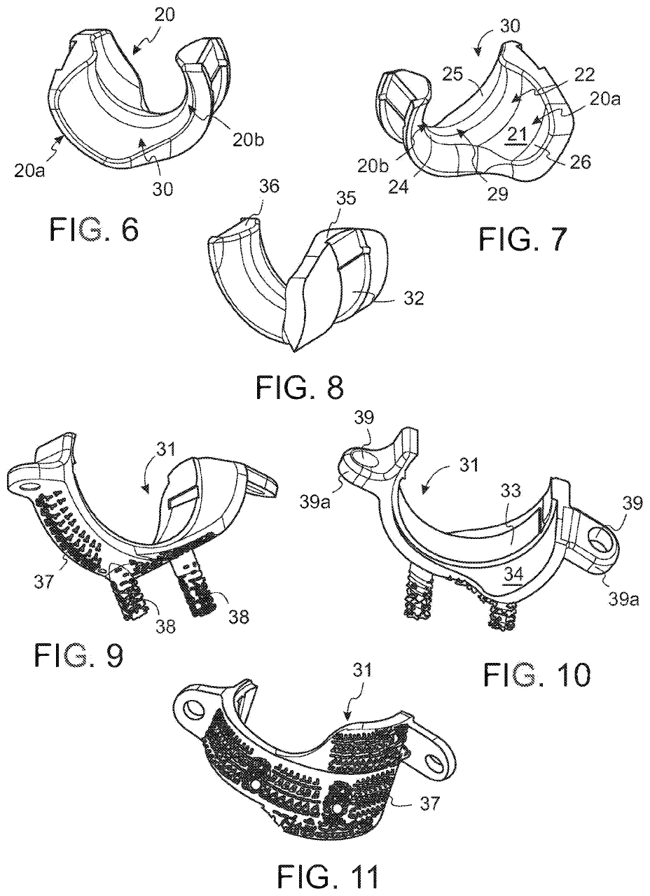

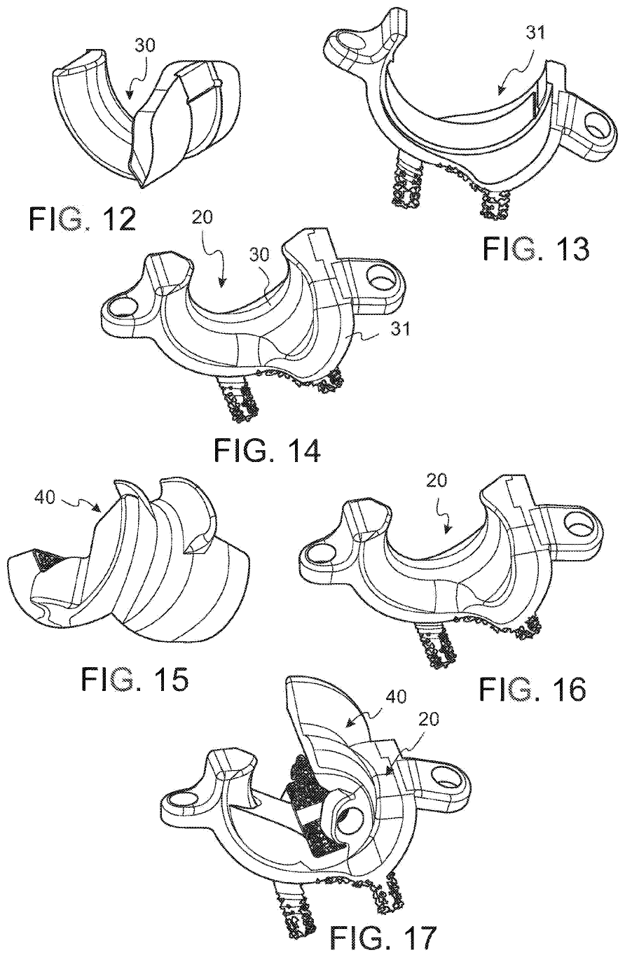

Referring to FIGS. 6 to 17, the radioulnar component 20 will now be further described. The radioulnar component 20 is generally like a tray for articulating with humeral component 40. As discussed above, the radioulnar component 20 has a contoured outer surface 21 defining an ulnar bearing surface 22 and an anconeal process bearing surface 24. The ulnar bearing surface 22 is configured for resurfacing the trochlear notch of a subject's ulna. The anconeal process bearing surface 24 is configured for resurfacing the anconeal process of the ulna.

The radioulnar component 20 comprises a bearing insert 30 and a supporting body 31, the insert having the contoured outer surface 21 thereon. The supporting body 31 may also be referred to as a tray. The bearing insert 30 and a supporting body 31 are shaped so that the bearing insert 30 can be received atop the supporting body 31. The bearing insert 30 and supporting body 31 are configured such that the bearing insert 30 can be coupled to the supporting body 31 via a snap fit (other means for securing the bearing insert to the supporting body can however be employed). The bearing insert 30 has a channel 32 on its back surface for receiving a protruding strip 33 on the top surface 34 of the supporting body 31 so that the bearing insert 30 can locate on and secure to the supporting body 31 via a snap fit.

The radioulnar component 20 is generally C-shaped in cross-section in the sagittal plane, the outer surface 21 being generally concave in the sagittal plane. The ulnar bearing surface 22 of the radioulnar component is shaped to articulate with the trochlear bearing surface 42 of the humeral component. The ulnar bearing surface 22 has medial and lateral recesses 25, 26 for articulating with the medial condylar bearing surface 43 and lateral condylar bearing surface 44 of the humeral component respectively. The medial and lateral recesses 25, 26 are separated by a ridge 29. The medial and lateral recesses 25, 26 are not identical, giving rise to an asymmetrical radioulnar component.

The ulnar bearing surface 22 is formed on a main portion 20a of the radioulnar component, the anconeal process bearing surface 24 being on a tail portion 20b which projects from the main portion 20a. The ulnar bearing surface 22 is a major or primary bearing surface of the radioulnar component, the anconeal process bearing surface 24 having a smaller surface area than the ulnar bearing surface 22, therefore the anconeal process bearing surface 24 forming a minor or secondary bearing surface.

The tail portion 20b projecting from the main portion 20a of the radioulnar component has a generally convex articular surface in the coronal plane, defining the anconeal process bearing surface 24. The anconeal process bearing surface 24 resurfaces the anconeal process of a subject and bears against the olecranon aperture boundary bearing surface 50 of the humeral component as the radioulnar component 20 as it enters and exits the olecranon aperture 10 of the humerus during extension and flexion of the elbow joint. The anconeal process bearing surface 24 is shaped to provide a ridge 29 which articulates with the curved track shape articulating surface of the olecranon aperture boundary bearing surface 50. The radioulnar component has an anterior end 35 and a posterior end 36, the radioulnar component being configured to be implanted on the radius and ulna with its anterior end 35 directed towards the anterior side of the humerus and towards coronoid process of the ulna and the posterior end 36 directed towards the posterior side of the humerus and anconeal process of the ulna. In the preferred embodiment in the figures, the ridge 29 runs from the anterior end 35 to the posterior end 36 of the radioulnar component. In alternative embodiments the ridge 29 may run at least part of the way from the anterior end 35 to the posterior end 36 of the radioulnar component, passing through both the ulnar bearing surface 22 and the anconeal process bearing surface 24. The ridge 29 forms an arc, the outer surface of which is concave in the sagittal plane. Referring to FIG. 26, when installed, the ridge 29 is received by groove 49 on the humeral component to aid articulation. The ridge runs through the ulnar bearing surface 22 and the anconeal process bearing surface 24. The ulnar bearing surface blends curvedly into the anconeal process bearing surface 24.

The bearing insert 30 of the radioulnar component 20 is preferably made of a polymeric material. In preferred embodiments the bearing insert 30 is made of polyether ether ketone (PEEK) or Ultra High Molecular Weight Polyethylene. The supporting body 31 is preferably made of metal. In preferred embodiments the supporting body 31 is made of titanium or titanium alloy.

Referring to FIGS. 9 to 11, the supporting body 31 of the radioulnar component has an inner surface 37 for seating against a prepared implantation site on the head of a subject's ulna. The inner surface 37 is generally convex in shape for seating against the concave bone surface at the implantation site on the ulna. The inner surface 37 is textured to promote attachment to the prepared bone surface. For example, the textured inner surface 37 may be porous or stippled. The textured surfaces of the joint assembly are preferably coated with hydroxyapatite (HA) to further promote bone attachment. Treatments other than surface texture and/or HA coating can of course be provided on the humeral component to promote attachment to the bone.

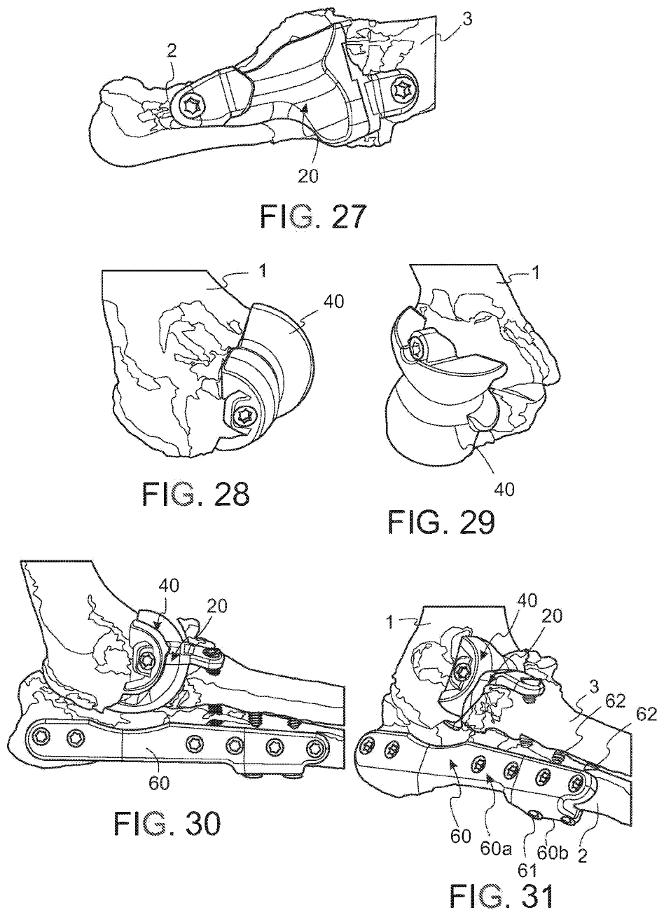

The supporting body component 31 has first and second small posts 38 projecting from its inner surface 37. The posts 38 are fixation posts arranged to secure the radioulnar component 30 in place on the humerus. Optionally, other fixation designs may be used, including a single post, more than two posts, ribs or blades. The supporting body comprises first and second through holes 39 at its distal and proximal ends respectively, each for receiving a screw 48 (visible in FIGS. 1, 30 and 31) for securing the radioulnar component to the bone. Optionally, other fixation designs may be used other than screw fixation. The proximal screw hole 39 is located in a wing 39a which projects from the proximal end 36 of the radioulnar component, and similarly the distal screw hole 39 is located in a wing 39a which projects from the distal end 35 of the radioulnar component, however screw holes may be located at other positions. Referring to FIGS. 27 and 31, the screw received by the proximal screw hole 39 is driven into the subject's ulna and the screw received by the distal screw hole is driven into the subject's radius. In this way, the radioulnar component 20 fuses the subject's radius and ulna bones.

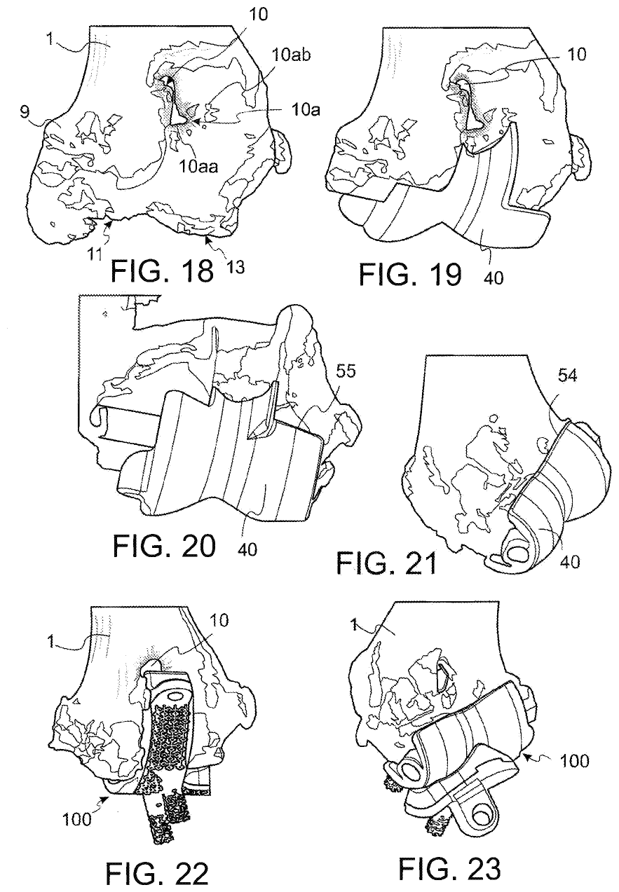

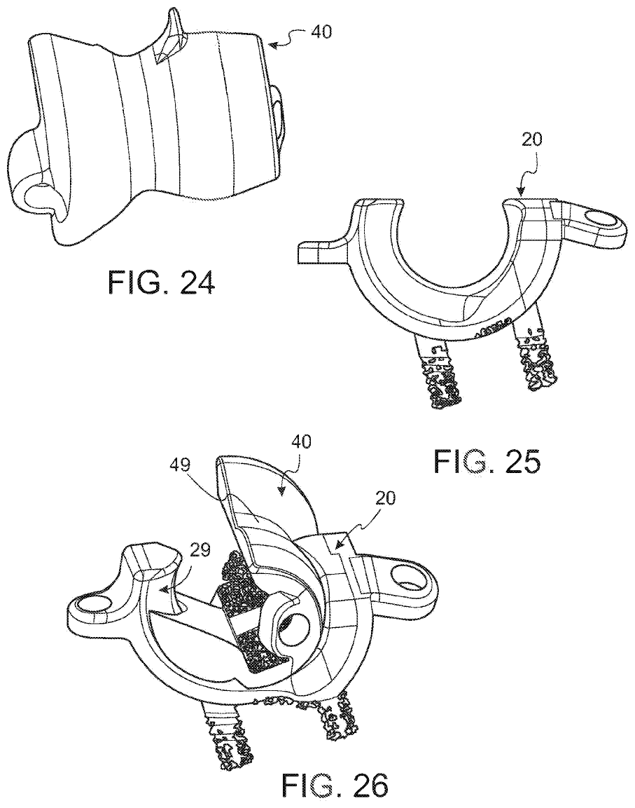

Referring to FIG. 17, the in preferred embodiments the C-shaped radioulnar component 20 has a small opening at the mouth of the ‘C’ sized so that the humeral component 40 snap fits to the radioulnar component 20. In preferred embodiments there is a small amount of clearance between the bearing surfaces of the humeral component and radioulnar component 20, therefore the assembly is described as semi-constrained.

Referring to FIGS. 1, 30 and 31, the total elbow replacement prosthesis assembly 100 further comprises an augmentation plate 60 for fixation to the subject's ulna. This plate is intended to be used optionally where the strength of ulna bone is weak. The augmentation plate 60 is preferably made of metal. The augmentation plate 60 has a large lateral portion 60a and a small posterior portion 60b at/near the distal end of the augmentation plate 60, the posterior portion 60b extending orthogonally from the lateral portion 60a. The lateral portion 60a secures to the lateral side of the ulna and the posterior portion 60b secures to the posterior side of the ulna. The augmentation plate 60 is provided with a plurality of screw holes 61, each for receiving a screw 62. In the present embodiment the lateral portion 60a has six screw holes and the posterior portion 60b has two screw holes, however it will be understood that other numbers of screw holes may be provided. The screws receivable by the screw holes 61 in the posterior portion 60b are long enough to pass through both the ulna and the radius, therefore strengthening the fusion of the radius and ulna together (the radius and ulna being fused by the radioulnar component 20 once implanted). Once installed, the augmentation plate 60 reinforces the subject's ulna to strengthen the bone against fracture and fuses the radius and ulna bones.

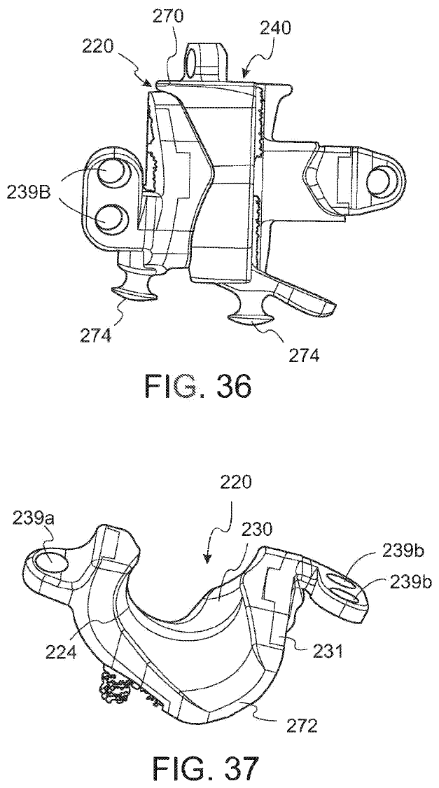

FIGS. 33 to 37 show an alternative embodiment of a TER prosthesis assembly 200. The TER prosthesis assembly 200 is similar to assembly 100 described above but with certain differences, which will now be described. Referring to FIG. 33, the humeral component 240 is shown. Like the humeral component 40, humeral component 240 has an olecranon aperture boundary bearing surface 250 having a lateral side wall 252 upstanding from the base 251. Unlike assembly 100, the humeral component 240 additionally has a medial condylar side wall 270 upstanding from the medial terminal end of the medial condylar bearing surface 243. Referring to FIG. 35, medial condylar side wall 270 substantially extends around the full circumferential extent of the medial condylar bearing surface 243.

Referring to FIG. 37, the radioulnar component 220 is alike in many respects the radioulnar component 20, but in particular the bearing insert 230 of the radioulnar component 220 has a medial overhanging portion 272 which is shaped to overhang the supporting body 231 of the radioulnar component 220 at the medial side. The medial overhanging portion 272 provides a medial bearing surface for bearing against the medial condylar side wall 270 of the humeral component in use. The medial condylar side wall 270 bearing against the medial overhanging portion 272 in use acts to counteract torsional forces caused by contact between lateral side wall 252 and the anconeal process bearing surface 224.

The TER prosthesis assembly 200 has screw holes for receiving screws for securing it to bone. Referring to FIG. 33, humeral component 240 has one screw hole 247a on its medial side. Referring to FIG. 35, it can be seen that the humeral component 240 has two screw holes 247b on its lateral side (as compared to humeral component 40, which has one screw hole 47 on its lateral side). Referring to FIG. 37, the radioulnar component 220 has one screw hole 239a at its proximal end and two screw holes 239b at its distal end (as compared to radioulnar component 20, which has one screw hole 39 at each end). The additional screw holes 247b and 239b on the humeral and radioulnar components provide additional fixation to counteract torsional forces experienced by the prosthesis assembly, thereby ensuring that the prosthesis assembly does not detach from the bone.

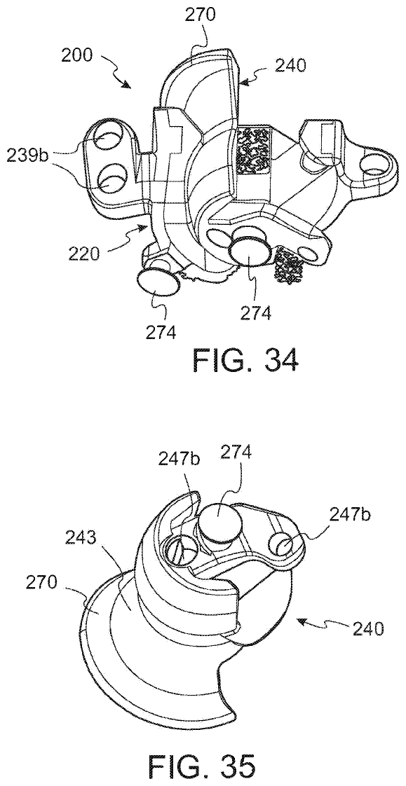

Referring to FIG. 34, the TER prosthesis assembly 200 additionally has a mushroom shaped button 274 on the lateral side of each of the humeral component 240 and the radioulnar component 220. The mushroom shaped buttons 274 are protrusions, each with a round flanged head projecting from a short cylindrical shaft. The buttons 274 act as anchors for one or more artificial ligaments. One or more artificial ligaments (not shown) can be arranged to each be looped around both of the buttons 274 so as to form an artificial ligament join between the two components and thereby provide further stability to the joint. The buttons 274 are located on the TER prosthesis assembly 200 at positions close to where the natural ligaments would be attached to the native bone. The shaft of the button 274 on the humeral component 240 aligns with the rotational axis of the TER prosthesis assembly 200, thus avoiding excessive forces being applied to the artificial ligament and the joint in use.

In order to install the TER prosthesis assembly 100, 200, recessed areas are prepared in the bone for receiving the prosthetic components. A recessed area for receiving the humeral component is prepared in the humerus to receive the humeral component and a recessed area in the radius and ulna is prepared to receive the radioulnar component. Suitable tools and guides may be provided to assist with preparing the bone before implantation. The thickness of each component from its back surface to its front surface is selected such that the trochlear bearing surface bears against the ulnar bearing surface in use and the anconeal process bearing surface bears against the olecranon aperture boundary surface in use, when the joint is in full extension, taking into account the depth of the respective recess prepared in the bone.

A plurality of different sized and/or shaped humeral components, radioulnar components and augmentation plates can be provided so components that are shaped/sized to suit the anatomy of the subject can be selected for installation. Alternatively the parts of the total elbow replacement prosthesis assembly may be custom made to suit the anatomy of a particular subject.

It will be understood that changes may be made in the details of the invention without departing from the spirit of the invention, especially as defined in the following claims.

1. A total elbow replacement prosthesis comprising:
a radioulnar component having an ulnar bearing surface and an anconeal process bearing surface,
a humeral component having a trochlear bearing surface, the trochlear bearing surface being configured for bearing against the ulnar bearing surface,
the humeral component further comprising an olecranon aperture boundary bearing surface, the anconeal process bearing surface being configured for bearing against the olecranon aperture boundary bearing surface.

2. A total elbow replacement prosthesis according to claim 1, wherein the olecranon aperture boundary bearing surface is shaped to resurface at least a portion of the lateral boundary of the olecranon aperture of a subject.

3. A total elbow replacement prosthesis according to claim 1, wherein the olecranon aperture boundary bearing surface comprises a lateral side wall upstanding from the humeral component.

4. A total elbow replacement prosthesis according to claim 1, wherein the olecranon aperture boundary bearing surface comprises a base and a lateral side wall upstanding from the base, the olecranon aperture boundary bearing surface being configured such that the base blends into the lateral side wall.

5. A total elbow replacement prosthesis according to claim 1, wherein the trochlear bearing surface is comprised on a main portion of the humeral component, the main portion comprising an elongate member with an outer surface of substantially C-shaped cross-section, the radius of the outer surface of the main portion varying along the elongate axis of the trochlear bearing surface.

6. A total elbow replacement prosthesis according to claim 1, wherein the trochlear bearing surface is comprised on a main portion of the humeral component, the main portion being an elongate member with an outer surface, the outer surface of the main portion being substantially bobbin shaped.

7. A total elbow replacement prosthesis according to claim 1, wherein the trochlear bearing surface comprises a medial condylar bearing surface and a lateral condylar bearing surface, each for bearing against a corresponding depression in the ulnar bearing surface.

8. A total elbow replacement prosthesis according to claim 1, wherein the humeral component is stemless.

9. A total elbow replacement prosthesis according to claim 1, wherein the humeral component has an outer surface which is generally convex in the sagittal plane.

10. A total elbow replacement prosthesis according to claim 1, wherein the olecranon aperture boundary bearing surface of the humeral component has an outer surface which is generally concave in the coronal plane.

11. A total elbow replacement prosthesis according to claim 1, wherein the radioulnar component has an outer surface which is generally concave in the sagittal plane.

12. A total elbow replacement prosthesis according to claim 1, wherein the ulnar bearing surface blends into the anconeal process bearing surface.

13. A total elbow replacement prosthesis according to claim 1, wherein the radioulnar component comprises means for attaching it to a subject's ulna bone and to the subject's radius bone.

14. A total elbow replacement prosthesis according to claim 1, the prosthesis further comprising an augmentation plate for attachment to a subject's radius and ulna.

15. A method of manufacturing a total elbow replacement prosthesis according to claim 1, the method comprising:
making a radioulnar component having an ulnar bearing surface and an anconeal process bearing surface,
making a humeral component having a trochlear bearing surface, the trochlear bearing surface being configured for bearing against the ulnar bearing surface,
the humeral component further comprising an olecranon aperture boundary bearing surface, the anconeal process bearing surface being configured for bearing against the olecranon aperture boundary bearing surface.
